(19) United States
(12) Patent Application Publication
(10) Pub. No.: US 2022/0118431 A1
(43) Pub. Date: Apr. 21, 2022

(54) BISMUTH-CONTAINING CATALYST COMPRISING AT LEAST ONE AROMATIC SUBSTITUENT

(71) Applicant: BASF Coatings GmbH, Münster (DE)

(72) Inventors: Peter Hoffmann, Münster (DE); Emre Levent, Münster (DE); Fabian Dielmann, Münster (DE)

(21) Appl. No.: 17/427,761

(22) PCT Filed: Jan. 24, 2020
(86) PCT No.: PCT/EP2020/051793

§ 371 Date: Aug. 2, 2021

(30) Foreign Application Priority Data
Feb. 7, 2019 (EP) 19155916.0

(51) Int. Cl. B01J 31/04; B01J 37/04; B01J 37/08; C07C 269/02

(52) U.S. Cl. CPC B01J 31/04; B01J 37/04; B01J 2531/54; C07C 269/02; B01J 37/08

(57) ABSTRACT

Described herein is a bismuth-containing catalyser, which is defined by a general formula (I). The bismuth-containing catalyser includes at least one radical R1, which includes a carboxyl fragment according to the general formula (II), where a first carbon atom (α-carbon) is bonded to the carbon atom of the carboxyl group, which in turn is directly substituted with at least one aromatic system. Also described herein is a method for preparing the bismuth-containing catalyser and a method of using the bismuth-containing catalyser for preparing compounds including a urethane group.

The invention relates to a bismuth-containing catalyser as such, which is defined by the general formula (I) detailed in the subsequent text. The bismuth-containing catalyser comprises at least one radical R¹, which comprises a carboxyl fragment according to the general formula (II), wherein a first carbon atom (α-carbon) is bonded to the carbon atom of the carboxyl group, which in turn is directly substituted with at least one aromatic system according to the invention. The present invention further relates to a method for preparing a bismuth-containing catalyser of this kind and also to the use of such a bismuth-containing catalyser for preparing compounds comprising a urethane group.

WO 2018/069018 relates to a coating composition system comprising the components (A) to (C) and optionally further components. The component (A) is at least one polyhydroxyl group-containing compound and the component (B) is at least one polyisocyanate-containing compound.

In contrast, the component (C) is a catalyser comprising at least two salts of an aliphatic monocarboxylic acid having at least 4 carbon atoms. In this case, the metal component of the first salt is bismuth (Bi), while the second salt comprises magnesium (Mg), sodium (Na), potassium (K) or calcium (Ca) as metal component. The coating composition system according to WO 2018/069018 may be configured according to a first option such that all components are present separately from one another, i.e. the individual components are not mixed with one another, whereas according to a second option of the corresponding coating composition system, the respective components can also be present completely or at least partially mixed with one another.

U.S. Pat. No. 4,895,827 discloses a catalyser in the form of a metal salt, in which the catalyser is a constituent of a heat-sensitive color-forming composition which, in addition to the catalyser, comprises a chromogenic material comprising an acidic developer and a suitable binder. The metal salt may comprise different metals as central metal atom/metal ion comprising, for example, zinc, tin, aluminum or nickel. The corresponding metal salt comprises organic compounds as ligands which, in addition to a carboxyl group, also comprise aromatic fragments and vinyl groups. However, bismuth-containing catalysers are not disclosed in U.S. Pat. No. 4,895,827.

JP-A 58 87 087 discloses the use of a multivalent metal salt of diphenylacetic acid, especially diphenylzinc acetate, as colorants, wherein a water-proof colored picture can be produced. However, bismuth-containing metal salts or the use of metal salts for preparing a compound comprising a urethane bond are not disclosed in JP-A 58 87 087.

The preparation of compounds comprising a urethane group (urethane bond) has likewise been known for a long time.

A compound having a urethane group is generally obtained if a compound comprising an isocyanate group is reacted with a compound comprising an hydroxyl group. The reaction generally takes place in the presence of a catalyser. Although tin-containing catalysers exhibit very high activity in such reactions, the use of such tin-containing catalysers, especially alkyltin compounds, should be avoided owing to their (very high) toxicity.

The object of the present invention, therefore, was to provide a novel catalyser which can be used for preparing compounds comprising a urethane group.

The object is achieved by a bismuth-containing catalyser of the general formula (I)

((R¹)⁻)ₓ((R²)⁻)_{y}((X)⁻)_{z}(Bi)³⁺ (I)
in which the variables are defined as follows:
R¹ is mutually independently a radical of the general

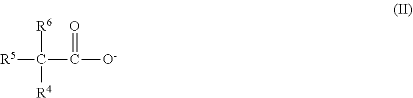

and x is 1, 2 or 3;
R² is mutually independently a radical of the general formula (III)

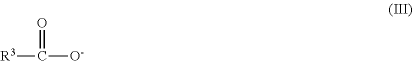

and y is 0, 1 or 2;
X is mutually independently hydroxyl, halogen, carbonate, hydrogencarbonate or R⁷ and z is 0, 1 or 2;
wherein the sum total of x, y and z is equal to 3;
R³ is unsubstituted or at least monosubstituted C₁-C₃₀-alkyl, C₆-C₁₄-aryl or C₇-C₃₀-aralkyl,
   wherein the substituents are selected from hydroxyl, halogen, carboxyl, —CF₃, —NH₂, C₁-C₆-alkoxy, C₁-C₃₀-alkyl or C₆-C₁₄-aryl and the alkyl and aryl fragments of these substituents may in turn be at least monosubstituted by hydroxyl, halogen, —CF₃, —NH₂ or C₁-C₆-alkoxy,
   and wherein the carbon atom of the radical R³ bonded directly to the carbon atom of the carboxyl group of the general formula (III) does not comprise any unsubstituted or at least monosubstituted C₆-C₁₄-aryl as substituent.
R⁴, R⁵ and R⁶ are mutually independently unsubstituted or at least monosubstituted C₁-C₃₀-alkyl, C₆-C₁₄-aryl or C₇-C₃₀-aralkyl,
   wherein the substituents are selected from hydroxyl, halogen, carboxyl, —CF₃, —NH₂, C₁-C₆-alkoxy, C₁-C₃₀-alkyl or C₆-C₁₄-aryl and the alkyl and aryl fragments of these substituents may in turn be at least monosubstituted by hydroxyl, halogen, —CF₃, —NH₂ or C₁-C₆-alkoxy,
   and wherein at least one of the radicals R⁴, R⁵ or R⁶ is unsubstituted or at least monosubstituted C₆-C₁₄-aryl,
R⁷ is unsubstituted or at least monosubstituted C₁-C₃₀-alkyl, C₆-C₁₄-aryl or C₇-C₃₀-aralkyl,
   wherein the substituents are selected from hydroxyl, halogen, carboxyl, —CF₃, —NH₂, C₁-C₆-alkoxy, C₁-C₃₀-alkyl or C₆-C₁₄-aryl and the alkyl and aryl fragments of these substituents may in turn be at least monosubstituted by hydroxyl, halogen, —CF₃, —NH₂ or C₁-C₆-alkoxy.

The bismuth-containing catalysers according to the invention are characterized in that, inter alia, the use of toxic tin-containing catalysers in the production of compounds comprising a urethane group can be avoided. The bismuth-containing catalysers according to the invention have a comparable catalytic activity as known representatives of the effective, on the one hand catalytically active, but on the other hand toxic, tin-containing catalysers.

However, the catalytic activity of the bismuth-containing catalysers according to the invention is better than the corresponding catalytic activity of the bismuth- or zinc-containing catalysers already known.

Moreover, the bismuth-containing catalysers according to the invention exhibit improved hydrolytic stability compared to bismuth-containing catalysers already known. For example, low amounts of water can already be sufficient in order to significantly or fully reduce the catalytic activity of bismuth-containing catalysers based on pure alkyl ligands, such as laurate-containing bismuth catalysers. Whereas such catalysers known from the prior art start to precipitate on contact with water, the bismuth-containing catalysers according to the invention are much more stable. Owing to the increased stability to hydrolysis—and thus also storage stability—the bismuth-containing catalysers according to the invention exhibit their improved catalytic properties over a much longer time period.

Furthermore, in the case of the bismuth-containing catalysers according to the invention, it is also not required that the catalyser must be present as a salt of the corresponding acid. The bismuth-containing catalysers according to the invention can thus be used without the presence of the corresponding acid at high catalytic activity in order to form compounds having urethane groups.

Advantageous properties are then already obtained in the catalysers according to the invention if in at least one of the substituents/ligands of the bismuth central atom, at least one aromatic system, especially a phenyl group, is directly substituted on the first carbon atom which is bonded to the carbon atom of the carboxyl group of the corresponding ligands. In accordance with the invention, this first carbon atom is referred to as the α-carbon (or in part also in chemical nomenclature). Known examples for this purpose from chemical nomenclature are α-amino acids, where the α-C atom is the carbon atom to which the amino group and the carboxyl group are attached. Specific examples for this numbering from the field of amino acids are β-alanine and gamma-aminobutyric acid. In chemical nomenclature, the carbonyl carbon is sometimes also counted and referred to as position 1. Accordingly, said first carbon atom directly adjacent to the carbon atom of the carboxyl group is sometimes also referred to as position 2 in chemical nomenclature. Accordingly, in accordance with the invention, at least one aromatic substituent, especially at least one phenyl substituent, is located on the α-carbon (atom) or in the 2-position, in the latter case based on the respective whole ligands taking carboxyl groups into account.

The said carboxyl group of this substituent is located (spatially speaking) in proximity to the bismuth central atom of the bismuth-containing catalyser. The bismuth-containing catalysers according to the invention are represented as salts, wherein the bismuth central atom of the bismuth-containing catalyser according to the invention is represented as a (triple positively charged) cation of the corresponding salt (see for example the general formula (I)). The corresponding substituents/ligands of the bismuth-containing catalyser, which are represented by the substituents/radicals R¹, R² and X in the general formula (I) detailed above, form the corresponding anion components of the bismuth-containing catalyser in this salt representation. Each of these so-called substituents/ligands is singly negatively charged. As detailed below, the two substituents R¹ and R² each comprise a carboxyl group. In general, the negative charge in the corresponding substituents/ligands of said carboxyl group is localized and/or the corresponding carboxyl group is located in spatial proximity to the (positively charged) bismuth central atom.

From a scientific standpoint however, it is also tenable, in place of the salt notation used in the context of the present application for the bismuth-containing catalysers according to the invention, to select a notation/representation in which a chemical bond between the bismuth central atom and the three ligands R¹, R² and X according to general formula (I) is completely or at least partially formed in each case. Expressed in other words, this means that the bismuth central atom is not present as a positively charged cation and the corresponding ligands are also not present as negatively charged anions, but rather the corresponding charge form a chemical bond between the corresponding ligands on the one hand and the bismuth central atom on the other hand. In the context of the present invention, the bismuth-containing catalysers disclosed according to the invention therefore also describe such a definition that is not based on a salt.

The catalytic activity and/or stability to hydrolysis/storage stability of the bismuth-containing catalysers according to the invention is further improved if at least two aromatic substituents, especially phenyl substituents, are presently directly on the at least one ligand on the first carbon atom (α-carbon atom/2-position), which in turn is bonded to the carbon atom of the carboxyl group. If three aromatic substituents, especially phenyl substituents, are bonded directly to said α-carbon atom, very catalytically active catalysers are also obtained, but their stability to hydrolysis (ostensibly with regard to heat) generally decreases somewhat in comparison to bismuth-containing catalysers according to the invention, in which precisely two aromatic systems, especially two phenyl groups, are present on at least one ligand on said α-carbon atom (2-position). The higher the catalytic activity and/or stability to hydrolysis of the bismuth-containing catalysers according to the invention, the higher the number of said radicals R¹ in the bismuth-containing catalyser according to the invention according to general formula (I). The best results are then achieved in this context, if the bismuth-containing catalyser according to the invention has three (identical or different) radicals R¹ according to the definition above, i.e. when the variable x=3. Particularly preferred catalysers of this kind are also shown below by the general formula (Ia).

In the context of the present invention, definitions such as C₁-C₃₀-alkyl, such as defined, for example, for the radical R⁴ in formula (II) above, signifies that this substituent (radical) is an alkyl radical having a carbon atom number of 1 to 30, wherein substituents optionally present are not taken into consideration in the carbon atom number. The alkyl radical may be either linear or branched as well as optionally cyclic. Alkyl radicals having both a cyclic and a linear component also fall under this definition. The same applies to other alkyl radicals such as a C₁-C₆-alkyl radical or a C₁-C₁₂-alkyl radical for example. Examples of alkyl radicals are methyl, ethyl, n-propyl, sec-propyl, n-butyl, sec-butyl, isobutyl, 2-ethylhexyl, tertiary-butyl (tert-Bu/t-Bu), pentyl, hexyl, heptyl, cyclohexyl, octyl, nonyl or decyl.

In the context of the present invention, the term “aryl” or the term “C₆-C₁₄-aryl”, as defined, for example, for the radical R⁴ in formula (II) above, signifies that the substituent (radical) is an aromatic system. The corresponding aromatic system has a carbon atom number of 6 to 14, wherein substituents optionally present are not taken into consideration in the carbon atom number. The aromatic system may be a monocyclic, bicyclic or optionally polycyclic aromatic system. In the case of bicyclic or polycyclic aromatic systems, individual rings may optionally be fully or partially saturated. Preferably, all rings of the corresponding aromatic systems are fully unsaturated. Preferred examples of aryl are phenyl, naphthyl or anthracyl, especially phenyl.

In the context of the present invention, the definition “C₇-C₃₀-aralkyl”, as defined for example for the radical R⁴ in formula (II) above, signifies that the substituent (radical) comprises an alkyl radical (such as C₁-C₆-alkyl according to the definitions above), wherein this alkyl radical is in turn substituted by an aryl radical (according to the definitions above). The corresponding aralkyl substituent has a carbon atom number of 7 to 30, wherein substituents optionally present are not taken into consideration in the carbon atom number. The alkyl radical itself present therein may be either linear or branched as well as optionally cyclic.

In the context of the present invention, the term “C₁-C₆-alkoxy”, as defined for example as (additional) substituent of the radical R⁴ in formula (II) above, signifies that it is a substituent (radical) in this case which is derived from an alcohol. The corresponding substituent thus comprises an oxygen fragment (—O—), which is in turn linked to an alkyl radical, such as C₁-C₆-alkyl (according to the definitions above). The alkyl radical itself may be either linear or branched as well as optionally cyclic.

In the context of the present invention, the term “halogen”, such as defined for example for the radical X in formula (I) above, signifies that the substituent (radical) is fluorine, chlorine, bromine or iodine, X preferably being fluorine or chlorine, particularly preferably chlorine.

In the context of the present invention, the term “unsubstituted or at least monosubstituted C₁-C₃O-alkyl, C₆-C₁₄-aryl or C₇-C₃₀-aralkyl”, such as defined for example for the radical R⁴ in formula (II) above, signifies that each of the in total three substituents (radicals) detailed corresponding to their definitions already specified above may be present either in unsubstituted form or have at least one further substituent (monosubstituted). If one or more substituents are present (for example disubstituted, trisubstituted or even higher substituted), the appropriate substituents are selected independently of one another from the substituent groups specified in each case.

In the case of a disubstituted C₆-C₁₄-aryl for example, the corresponding aryl unit, such as phenyl for example, may be substituted for example by an hydroxyl and a C₁-C₃₀-alkyl substituent, such as methyl or ethyl. Alkyl or aryl fragments may themselves in turn comprise at least one additional substituent according to the definitions stated. The substitution may be at any desired position of the corresponding fragment.

Provided that an appropriate radical, such as R⁴ for example, owing to the definition of, for example, formula (I), may occur two or more times in connection with formula (II), the individual radicals R⁴ may be selected completely independently of one another according to the respective definitions. For example, if in the general formula (I) the variable x=3, the radical R¹ occurs in total three times in the general formula (I). The radical R¹, which in turn comprises the radical R⁴, can therefore be present three times independently of one another in this scenario. The individual radicals R¹ can therefore be configured differently according to their basic definition. Thus, it is feasible that a first radical R¹, with regard to the constituent of the radical R⁴ necessarily present therein, has a definition other than the corresponding second and/or third radical R¹. Unless otherwise stated in the following text, this logically applies also for all other radicals, such as R², R³, R⁵ and/or R⁶.

Unless otherwise specified in the following description, the respective definitions of the radicals R¹ to R⁷ are in each case the preferred unsubstituted definitions.

The present invention is further specified hereinbelow.

The present invention firstly relates to a bismuth-containing catalyser of the general formula (I)

((R¹)⁻)ₓ((R²)⁻)_{y}((X)⁻)_{z}(Bi)³⁺ (I)
in which the variables are defined as follows:
R¹ is mutually independently a radical of the general formula (II)

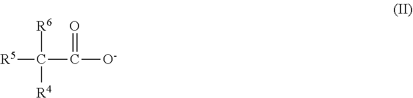

and x is 1, 2 or 3;
R² is mutually independently a radical of the general formula (III)

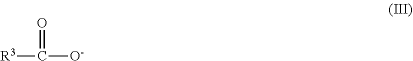

and y is 0, 1 or 2;
X is mutually independently hydroxyl, halogen, carbonate, hydrogencarbonate or R⁷ and z is 0, 1 or 2;
wherein the sum total of x, y and z is equal to 3;
R³ is unsubstituted or at least monosubstituted C₁-C₃₀-alkyl, C₆-C₁₄-aryl or C₇-C₃₀-aralkyl,
   wherein the substituents are selected from hydroxyl, halogen, carboxyl, —CF₃, —NH₂, C₁-C₆-alkoxy, C₁-C₃₀-alkyl or C₆-C₁₄-aryl and the alkyl and aryl fragments of these substituents may in turn be at least monosubstituted by hydroxyl, halogen, —CF₃, —NH₂ or C₁-C₆-alkoxy,
   and wherein the carbon atom of the radical R³ bonded directly to the carbon atom of the carboxyl group of the general formula (III) does not comprise any unsubstituted or at least monosubstituted C₆-C₁₄-aryl as substituent.
R⁴, R⁵ and R⁶ are mutually independently unsubstituted or at least monosubstituted C₁-C₃O-alkyl, C₆-C₁₄-aryl or C₇-C₃₀-aralkyl,
   wherein the substituents are selected from hydroxyl, halogen, carboxyl, —CF₃, —NH₂, C₁-C₆-alkoxy, C₁-C₃₀-alkyl or C₆-C₁₄-aryl and the alkyl and aryl fragments of these substituents may in turn be at least monosubstituted by hydroxyl, halogen, —CF₃, —NH₂ or C₁-C₆-alkoxy, and wherein at least one of the radicals R⁴, R⁵ or R⁶ is unsubstituted or at least monosubstituted C₆-C₁₄-aryl,
R⁷ is unsubstituted or at least monosubstituted C₁-C₃₀-alkyl, C₆-C₁₄-aryl or C₇-C₃₀-aralkyl,
   wherein the substituents are selected from hydroxyl, halogen, carboxyl, —CF₃, —NH₂, C₁-C₆-alkoxy, C₁-C₃₀-alkyl or C₆-C₁₄-aryl and the alkyl and aryl fragments of these substituents may in turn be at least monosubstituted by hydroxyl, halogen, —CF₃, —NH₂ or C₁-C₆-alkoxy.

In connection with the radicals (substituents/ligands) present in the general formula (I), particularly the necessary radical R¹ and the optional radical R², it should be noted that the further/exact chemical definition of these radicals R¹ or R² is a result of the radicals R⁴ to R⁶ of the general formula (II) with respect to the radical R¹ and is a result of the radical R³ of the general formula (III) with respect to the radical R². In accordance with the invention, the radical R² is chemically always defined differently than the radical R¹. Expressed in other words, this signifies that a specific radical R² or R³ in accordance with general formula (III) cannot fall under the corresponding definition of a radical R¹ or the radicals R⁴ to R⁶ according to general formula (II). This results in particular from the fact that, in the chemical definition of the radical R² in accordance with general formula (III), a radical R³ is present which is defined such that the radical R³ is directly bonded to the carbon atom of the carbon atom of the corresponding carboxyl group of the general formula (III), no unsubstituted or at least monosubstituted C₆-C₁₄-aryl, especially no phenyl, can be present as direct substituent. In contrast, such a substituent is necessarily present for at least one of the radicals R⁴, R⁵ or R⁶ in accordance with the general formula (II) in connection with the radical R¹. Accordingly, it is excluded that a substituent falling under the definition of the radical R² in accordance with general formula (III) can at the same time fall under the corresponding definition of the radical R¹ in accordance with general formula (II).

The radical R¹ in accordance with general formula (II) is preferably defined according to the invention such that R⁴, R⁵ and R⁶ are mutually independently unsubstituted or at least monosubstituted C₁-C₁₂-alkyl or C₆-C₁₄-aryl, wherein the substituents are selected from hydroxyl, chlorine, —CF₃ and C₁-C₆-alkyl, and wherein at least one of the radicals R⁴, R⁵ or R⁶ is unsubstituted or at least monosubstituted C₆-C₁₄-aryl. C₆-C₁₄-aryl is preferably phenyl, especially unsubstituted phenyl.

Furthermore, it is preferred in accordance with the invention that the radicals R⁴ to R⁶ in the general formula (II) are defined as follows:
i) R⁴ is unsubstituted or at least monosubstituted phenyl, wherein the substituents are selected from hydroxyl, chlorine, —CF₃ and C₁-C₆-alkyl,
ii) R⁵ is unsubstituted or at least monosubstituted phenyl or C₁-C₁₂-alkyl, wherein the substituents are selected from hydroxyl, chlorine, —CF₃ and C₁-C₆-alkyl, and
iii) R⁶ is unsubstituted or at least monosubstituted phenyl or C₁-C₁₂-alkyl, wherein the substituents are selected from hydroxyl, chlorine, —CF₃ and C₁-C₆-alkyl.

With respect to the radical R² according to general formula (III), it is preferable that the radical R³ present therein is unsubstituted or at least monosubstituted C₁-C₁₂-alkyl, wherein the substituents are selected from hydroxyl, chlorine or —CF₃.

With regard to the radical X, it is preferred in accordance with the invention that this is hydroxyl, chlorine or R⁷ and R⁷ is unsubstituted or at least monosubstituted C₁-C₁₂-alkyl or C₆-C₁₄-aryl, wherein the substituents are selected from hydroxyl, chlorine, —CF₃ and C₁-C₆-alkyl.

In the bismuth-containing catalyser according to general formula (I) according to the invention, the radicals R¹, R² and X present in each case can be present in each case in any combination. A precondition in this case however is that x is at least 1, i.e. at least one radical R¹ is present in the bismuth-containing catalyser according to the invention according to general formula (I). Furthermore, the sum total of x, y and z is 3, the total number of ligands or negative charges is 3, so that in total charge neutrality is present with respect to the 3-fold positively charged bismuth central atom. If individual ligands/radicals such as R¹, R² or X occur repeatedly, these can have the same or different definitions according to the respective basic definition.

In the context of general formula (I), the following scenarios are preferred in accordance with the invention, wherein
i) x is equal to 2 or 3, y is equal to 0 or 1 and z is equal to 0 or 1, or
ii) x is equal to 3 and y and z are each equal to 0, or
iii) x is equal to 2, y is equal to 0 and z is equal to 1, or
iv) x is equal to 2, y is equal to 1 and z is equal to 0,
   wherein preferably x is equal to 3 and y and z are each equal to 0.

In a preferred embodiment of the present invention, the bismuth-containing catalyser according to the general formula (I) and (II) is defined as follows:
i) R⁴ is unsubstituted or at least monosubstituted phenyl, wherein the substituents are selected from hydroxyl, chlorine, —CF₃ and C₁-C₆-alkyl,
ii) R⁵ is unsubstituted or at least monosubstituted phenyl or C₁-C₁₂-alkyl, wherein the substituents are selected from hydroxyl, chlorine, —CF₃ and C₁-C₆-alkyl,
iii) R⁶ is unsubstituted or at least monosubstituted C₁-C₁₂-alkyl, wherein the substituents are selected from hydroxyl, chlorine and —CF₃, and
iv) x is equal to 3 and y and z are each equal to 0.

In a further preferred embodiment of the present invention, the bismuth-containing catalyser is defined as follows:
i) R⁴ and R⁵ are each phenyl,
ii) R⁶ is C₁-C₁₂-alkyl, preferably C₆-C₁₀-alkyl, especially C₈-alkyl, and
iii) x is equal to 3 and y and z are each equal to 0.

In a particularly preferred embodiment of the present invention, the bismuth-containing catalyser is defined by the general formula (Ia):

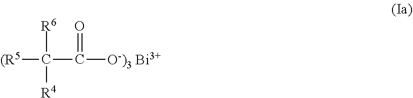

wherein the radicals R⁴ to R⁶ mutually independently correspond to the definitions described above. As evident from formula (Ia) compared to the general formula (I), the catalyser according to the invention in this embodiment has in total three radicals R¹ according to general formula (I). Each of these in total three radicals R¹ in each case comprises one radical R⁴, one radical R⁵ and one radical R⁶. In each of these three radicals R¹, the corresponding definitions of the radicals R⁴ to R⁶ can be the same or different from one another. However, it is preferred in accordance with the invention that each of the total of three radicals R⁴ are the same, each of the total of three radicals R⁵ are the same and each of the total of three radicals R⁶ are the same.

Furthermore, in the context of the general formula (Ia), it is preferable that
i) R⁴ is unsubstituted or at least monosubstituted phenyl, wherein the substituents are selected from hydroxyl, chlorine, —CF₃ and C₁-C₆-alkyl, ii) R⁵ is unsubstituted or at least monosubstituted phenyl or C₁-C₁₂-alkyl, wherein the substituents are selected from hydroxyl, chlorine, —CF₃ and C₁-C₆-alkyl, and
iii) R⁶ is unsubstituted or at least monosubstituted C₁-C₁₂-alkyl, wherein the substituents are selected from hydroxyl, chlorine and —CF₃.

In the context of the general formula (Ia), it is even more preferable that
i) R⁴ and R⁵ are each phenyl, and
ii) R⁶ is C₁-C₁₂-alkyl, preferably C₆-C₁₀-alkyl, especially C₈-alkyl.

The present invention therefore further relates also to a method for preparing a bismuth-containing catalyser of the general formula (I) or of the general formula (Ia) according to the definitions above. The method according to the invention for preparing such bismuth-containing catalysers can be carried out for example, in which
i) at least one compound of the general formula

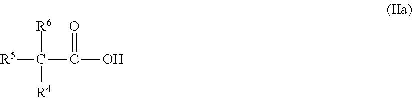

or a corresponding salt thereof,
wherein the radicals R⁴ to R⁶ are defined according to the embodiments/definitions above,
ii) optionally at least one compound of the general formula (IIIa)

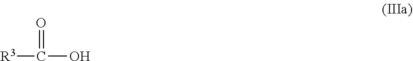

or a corresponding salt thereof,
wherein the radical R³ is defined according to the embodiments/definitions above,
iii) is reacted with at least one bismuth-containing compound selected from Bi₂O₃, bismuth carbonate, bismuth hydrogencarbonate, bismuth halide, Bi(C₆-C₁₄-aryl) 3, Bi(C₁-C₁₂-alkyl) 3 or metallic bismuth.

The reactants listed above, i.e. the acids according to the general formulae (IIa) or (IIIa) or the appropriate corresponding salts as such, are known to those skilled in the art. The corresponding salts used can be, for example, sodium, potassium or calcium salts. Optionally, instead of the aforementioned acids according to the general formulae (IIa) or (IIIa) or corresponding salts thereof as reactants, it is also possible to use corresponding carboxylic esters, for example a methyl or ethyl ester. Such carboxylic esters can be prepared by reacting the aforementioned acids or a corresponding salt thereof with a suitable alcohol, for example methanol or ethanol, optionally in the presence of a catalyser. The appropriate preparation methods of such carboxylic esters are known to a person skilled in the art.

In principle, any bismuth-containing compound can be used in the method according to the invention, which is suitable for the purpose of forming the bismuth central atom in the bismuth-containing catalyser of the general formula (I) according to the invention, by reaction with the appropriate compound according to the general formula (IIa) or optionally (IIIa). Bismuth-containing compounds as such are known to those skilled in the art. If, in accordance with the invention, a bismuth halide is used as bismuth-containing compound, it is preferably a chlorine-containing compound, especially BiCl₃. Any specific substituents/substitution patterns, such as the radicals R⁴ to R⁶ for example, may already be present in the corresponding reactant. Optionally, such substituents/substitution patterns can also be attached or completed even after the preparation process of a bismuth-containing catalyser according to the general formula (I) described above.

Preferably, the bismuth-containing compound is selected from Bi₂O₃, BiCl₃, Bi(C₆H₅)₃ or metallic bismuth.

The bismuth-containing catalysers according to the general formula (I) according to the invention are preferably prepared by reacting at least one compound of the general formula (IIa) and optionally at least one compound of the general formula (IIIa) with at least one bismuth-containing compound, wherein
i) the reaction is carried out under a protective atmosphere and/or in the presence of at least one solvent, especially toluene or tetrahydrofuran, and/or
ii) the reaction is conducted for at least 10 hours and/or at a temperature of at least 100° C., and/or
iii) following the reaction, volatile constituents are removed, the bismuth-containing catalyser is dried under reduced pressure and/or a recrystallization is carried out.

Furthermore, it is preferred in accordance with the invention that the at least one compound of the general formula (IIa) used as reactant in the method according to the invention is prepared from a corresponding compound according to the general formula (IIb), wherein the compounds according to the general formula (IIa) only differ from the corresponding compounds of the general formula (IIb) in that one or at most two radicals selected from R⁴, R⁵ and R⁶ is defined as H (hydrogen) in place of the definitions listed for the compounds according to general formula (IIa). This is preferably effected by reacting a corresponding compound (IIb), but in which R⁵ and/or R⁶ is H, with a lithium-containing compound, especially with n-butyllithium, and the intermediate obtained in this case is subsequently reacted with a haloalkane to introduce the radicals R⁵ and R⁶ to obtain a compound according to the general formula (IIa). Haloalkanes used can be, for example, 1-bromooctane or 1-bromopropane. This method variant is thus used in particular if a catalyser according to the invention in accordance with general formula (I) is intended to be prepared in which at least one of the radicals R⁴ to R⁶, preferably precisely one of these radicals, is a C₁-C₃₀-alkyl. Alternatively, it is also conceivable that, instead of a haloalkane, an appropriate haloaryl or haloaralkyl compound is used if, in the context of this method step, a fully or partially aromatic substituent is intended to be introduced into the corresponding compound (IIb). Preferably, the compound according to the general formula (IIb) used is 2-arylacetic acid, 2,2-diarylacetic acid, particularly 2-phenylacetic acid or 2,2-diphenylacetic acid, particularly preferably 2,2-diphenylacetic acid.

The present invention further relates to the use of at least one bismuth-containing catalyser according to the definitions above for preparing compounds comprising a urethane group.

The invention is illustrated hereinafter by examples.

### I) PREPARATION OF CATALYSERS ACCORDING TO THE INVENTION

### Ia) Precursor of Example 1: 2,2-Diphenyldecanoic Acid (dpdH)

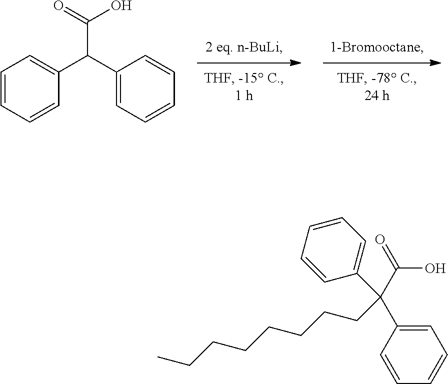 [SMILES: O=C(O)C(c1ccccc1)c1ccccc1 ; CCCCCCCCC(C(=O)O)(c1ccccc1)c1ccccc1]

2,2-Diphenylacetic acid (10.6 g; 48 mmol) are dissolved in 75 mL of dry tetrahydrofuran (THF) under a protective gas atmosphere (argon or nitrogen) in a 200 mL Schlenk flask and cooled to −15° C. A 1.6M solution of n-butyllithium in hexane (60 mL; 96 mmol) is then added with stirring and over a period of 30 minutes. The reaction solution is stirred at −15° C. for one hour and cooled to −78° C. for the addition of 1-bromooctane (8.3 mL; 48 mmol). Subsequently, the reaction solution is slowly warmed to room temperature and stirred for a further 24 hours.

For the work-up and purification of the 2,2-diphenyldecanoic acid, a saturated ammonium chloride solution (60 mL) is added to the reaction solution and stirred for 30 minutes. The aqueous phase is separated by means of a separating funnel and extracted with 3×25 mL of diethyl ether. The combined organic phases were dried over magnesium sulfate (MgSO₄). All volatile solvents are then removed under reduced pressure (1·10-3 mbar) and the resulting solid dried at 140° C. under reduced pressure (1·10⁻³ mbar) for 24 hours.

Characterization by ¹H-NMR, ¹³C-NMR, HRMS (high-resolution mass spectrometry), infra-red spectroscopy.

### Ib) Catalyser According to Example 1: Bi(2,2-Diphenyldecanoate)₃ (Bi(dpd)₃)

Triphenylbismuth (1.1 g; 2.5 mmol) and 2,2-diphenyldecanoic acid (2.43 g; 7.5 mmol) are initially charged under a protective gas atmosphere in a 25 mL three-necked flask equipped with stirrer bar, reflux condenser, thermometer and protective gas atmosphere inlet (argon or nitrogen). 12.5 mL of dry tetrahydrofuran or dry toluene (5 mL of solvent per 1 mmol of triphenylbismuth) are added to the reactants and the mixture is heated at 110° C. under a protective gas atmosphere for at least 16 hours. The reaction course is monitored by ¹H-NMR (nuclear magnetic resonance spectroscopy). After complete conversion of triphenylbismuth with formation of benzene, the reaction is terminated and cooled. All volatile solvents are then removed under reduced pressure (1·10⁻³ mbar) and the resulting solid dried at 60° C. under reduced pressure (1·10⁻³ mbar) for 24 hours. As required, the resulting compound is purified from toluene and hexane at −40° C. or by recrystallization from hot toluene.

Characterization by ¹H-NMR, ¹³C-NMR, C/H/N elemental analysis, infra-red spectroscopy.

### Ic) Catalyser According to Example 4: Bi(2,2-Diphenylpropionate)₃ (Bi(dpp)₃)

The catalyser Bi(dpp)₃ according to example 4 is prepared analogously to the catalyser Bi(dpd)₃ described above, in which no synthesis of the precursor is required since the corresponding propionic acid derivative is commercially available (from Sigma-Aldrich), which is reacted with triphenylbismuth to give the inventive catalyser Bi(dpp)₃.

### II) DETERMINATION OF THE CATALYTIC ACTIVITY OF EXAMPLES 1 TO 5

The respective catalytic activity of the individual working and comparative examples can be found in Table 1 below. The catalytic activity is tested by means of a reaction in which a compound is formed comprising a urethane group. For this purpose, 11 mmol of 2-ethylhexyl (6-isocyanatohexyl)carbamate (commercially available as Desmodur LD (3.3 mL)) are reacted with 11 mmol of n-butanol (1 mL) as reactants. The reaction was carried out in the presence of a solvent (2 mL of xylene) and the catalysers listed in Table 1 (with a content of 0.1 mol % catalyser based on the amount of bismuth) at a temperature of 60° C.

The isocyanate decrease and thus the formation of a urethane group are investigated by horizontal ATR-IR spectroscopy. For this purpose, 0.05 mL of the reaction solution are withdrawn at defined time intervals and investigated directly by spectroscopy. The conversion is determined by the relative decrease in intensity of the asymmetrical isocyanate stretching vibration at 2250-2285 cm-1. The starting content of free isocyanate of the reaction solution at room temperature in the absence of catalyser was determined. All IR spectra were normalized to the bands of the symmetrical and asymmetrical stretching vibrations of the CH₂ groups (3000-2870 cm-1).

The catalysers used are detailed in Table 1.

Example 1: Bi(dpd)₃, wherein dpd is 2,2′-diphenyl decanoate
Comparative example 2: DOTL (Dioctyltin dilaurate), commercially available catalyser
Comparative example 3: Bi(neo)₃, wherein neo is neodecanoate (commercially available catalyser under the name K-Kat XK651 (King Industries); 50% in neodecanoic acid having a metal content of 23%
Example 4: Bi(dpp)₃, wherein dpp is 2,2′-diphenyl propionate
Comparative example 5: without catalyser

TABLE 1

| | | | | | Bi(neo)₃/ | | | | No |
|---|---|---|---|---|---|---|---|---|---|
| Ex. | Bi (dpd)₃ | Comp. | DOTL | Comp. | XK651 | Ex. | Bi(dpp)₃ | Comp. | catalyser |
| 1 | NCO | 2 | NCO | 3 | NCO | 4 | NCO | 5 | NCO |
| t/ | degra- | t/ | degra- | t/ | degra- | t/ | degra- | t/ | degra- |
| min | dation | min | dation/% | min | dation/% | min | dation/% | min | dation/% |
| 0 | 0 | 0 | 0 | 0 | 0 | 0 | 0 | 0 | 0 |
| 1 | 43.58 | 1 | 58.03 | 5 | 18 | 5 | 33.14 | 10 | 14.5 |
| 2 | 83.97 | 3 | 95.6 | 10 | 29.11 | 10 | 55.9 | 20 | 22.15 |
| 3 | 97.800 | 5 | 99.6 | 15 | 40.77 | 15 | 76.92 | 30 | 28.04 |
| 4 | 100.000 | | | 20 | 53.12 | 20 | 90.11 | | |
| | | | | 25 | 67.135 | 25 | 97.5 | | |
| | | | | 30 | 80.04 | 30 | 100 | | |

As can be deduced from Table 1, the two catalysers according to the invention show, according to working example 1 and working example 4, a comparable catalytic activity to the known tin-containing catalysers according to comparative example 2. Tin-containing catalysers should be avoided however, owing to their considerable toxicity. The catalytic activity of working examples 1 and 4 is however distinctly improved in comparison to bismuth-containing catalysers according to the prior art (comparative example 3) or conducting the experiment wholly without catalyser (comparative example 5).

1. A bismuth-containing catalyser of a general formula (I)
((R¹)⁻)ₓ((R²)⁻)_{y}((X)⁻)_{z}(Bi)³⁺ (I)
in which the variables are defined as follows:
R¹ is mutually independently a radical of a general formula (II) 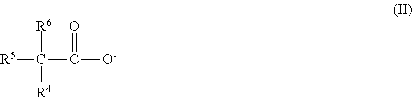
and x is 1, 2 or 3;
R² is mutually independently a radical of a general formula (III) 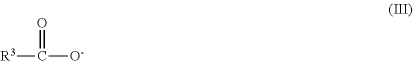
and y is 0, 1 or 2;
X is mutually independently hydroxyl, halogen, carbonate, hydrogencarbonate or R⁷ and z is 0, 1 or 2;
wherein the sum total of x, y and z is equal to 3;
R³ is unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂ or C₁-C₆-alkoxy,
and wherein the carbon atom of the radical R³ bonded directly to the carbon atom of the carboxyl group of the general formula (III) does not comprise any unsubstituted or at least monosubstituted C₆-C₁₄-aryl as substituent.
R⁴, R⁵ and R⁶ are mutually independently unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂ or C₁-C₆-alkoxy,
and wherein at least one of the radicals R⁴, R⁵ or R⁶ is unsubstituted or at least monosubstituted C₆-C₁₄-aryl,
R⁷ is unsubstituted or at least monosubstituted C₁-C₃N-alkyl, C₆-C₁₄-aryl or C₇-C₃₀-aralkyl,
wherein the substituents are selected from the group consisting of hydroxyl, halogen, carboxyl, —CF₃, —NH₂, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂ or C₁-C₆-alkoxy.

2. The bismuth-containing catalyser as claimed in claim 1, wherein
i) R⁴, R⁵ and R⁶ are mutually independently unsubstituted or at least monosubstituted C₁-C₁₂-alkyl or C₆-C₁₄-aryl, wherein the substituents are selected from the group consisting of hydroxyl, chlorine, —CF₃ and C₁-C₆-alkyl,
and wherein at least one of the radicals R⁴, R⁵ or R⁶ is unsubstituted or at least monosubstituted C₆-C₁₄-aryl, and/or
ii) R³ is unsubstituted or at least monosubstituted C₁-C₁₂-alkyl, wherein the substituents are selected from the group consisting of hydroxyl, chlorine and —CF₃, and/or
iii) X is hydroxyl, chlorine or R⁷ and R⁷ is unsubstituted or at least monosubstituted C₁-C₁₂-alkyl or C₆-C₁₄-aryl, wherein the substituents are selected from the group consisting of hydroxyl, chlorine, —CF₃ and C₁-C₆-alkyl.

3. The bismuth-containing catalyser as claimed in claim 1, wherein
i) x is equal to 2 or 3, y is equal to 0 or 1 and z is equal to 0 or 1, or
ii) x is equal to 3 and y and z are each equal to 0, or
iii) x is equal to 2, y is equal to 0 and z is equal to 1, or
iv) x is equal to 2, y is equal to 1 and z is equal to 0.

4. The bismuth-containing catalyser as claimed in claim 1, wherein
i) R⁴ is unsubstituted or at least monosubstituted phenyl, wherein the substituents are selected from the group consisting of hydroxyl, chlorine, —CF₃ and C₁-C₆-alkyl,
ii) R⁵ is unsubstituted or at least monosubstituted phenyl or C₁-C₁₂-alkyl, wherein the substituents are selected from the group consisting of hydroxyl, chlorine, —CF₃ and C₁-C₆-alkyl, and
iii) R⁶ is unsubstituted or at least monosubstituted phenyl or C₁-C₁₂-alkyl, wherein the substituents are selected from the group consisting of hydroxyl, chlorine, —CF₃ and C₁-C₆-alkyl.

5. The bismuth-containing catalyser as claimed in claim 1, wherein
i) R⁴ is unsubstituted or at least monosubstituted phenyl, wherein the substituents are selected from the group consisting of hydroxyl, chlorine, —CF₃ and C₁-C₆-alkyl,
ii) R⁵ is unsubstituted or at least monosubstituted phenyl or C₁-C₁₂-alkyl, wherein the substituents are selected from the group consisting of hydroxyl, chlorine, —CF₃ and C₁-C₆-alkyl,
iii) R⁶ is unsubstituted or at least monosubstituted C₁-C₁₂-alkyl, wherein the substituents are selected from the group consisting of hydroxyl, chlorine and —CF₃, and
iv) x is equal to 3 and y and z are each equal to 0.

6. The bismuth-containing catalyser as claimed in claim 1, wherein
i) R⁴ and R⁵ are each phenyl,
ii) R⁶ is C₁-C₁₂-alkyl, and
iii) x is equal to 3 and y and z are each equal to 0.

7. The bismuth-containing catalyser as claimed in claim 1, wherein the bismuth-containing catalyser is defined according to a general formula (Ia) 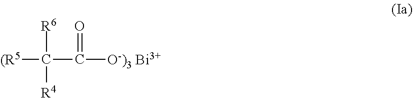
wherein R⁴, R⁵ and R⁶ are mutually independently unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂ or C₁-C₆-alkoxy,
and wherein at least one of the radicals R⁴, R⁵ or R⁶ is unsubstituted or at least monosubstituted C₆-C₁₄-aryl.

8. The bismuth-containing catalyser as claimed in claim 7, wherein each of the total of three radicals R⁴ are the same, each of the total of three radicals R⁵ are the same and each of the total of three radicals R⁶ are the same.

9. The bismuth-containing catalyser as claimed in claim 7, wherein
i) R⁴ is unsubstituted or at least monosubstituted phenyl, wherein the substituents are selected from the group consisting of hydroxyl, chlorine, —CF₃ and C₁-C₆-alkyl,
ii) R⁵ is unsubstituted or at least monosubstituted phenyl or C₁-C₁₂-alkyl, wherein the substituents are selected from the group consisting of hydroxyl, chlorine, —CF₃ and C₁-C₆-alkyl, and
iii) R⁶ is unsubstituted or at least monosubstituted C₁-C₁₂-alkyl, wherein the substituents are selected from the group consisting of hydroxyl, chlorine and —CF₃.

10. The bismuth-containing catalyser as claimed in claim 7, wherein
i) R⁴ and R⁵ are each phenyl, and
ii) R⁶ is C₁-C₁₂-alkyl.

11. A method for preparing a bismuth-containing catalyser of the general formula (I) as claimed in claim 1, comprising reacting
i) at least one compound of a general formula (IIa) 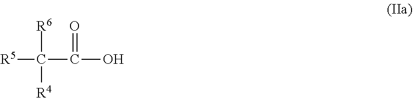
or a corresponding salt thereof,
wherein R⁴, R⁵ and R⁶ are mutually independently unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂ or C₁-C₆-alkoxy,
and wherein at least one of the radicals R⁴, R⁵ or R⁶ is unsubstituted or at least monosubstituted C₆-C₁₄-aryl,
ii) optionally at least one compound of a general formula (IIIa) 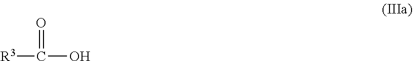
or a corresponding salt thereof,
wherein R³ is unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂ or C₁-C₆-alkoxy,
and wherein the carbon atom of the radical R³ bonded directly to the carbon atom of the carboxyl group of the general formula (IIIa) does not comprise any unsubstituted or at least monosubstituted C₆-C₁₄-aryl as substituent, and
iii) at least one bismuth-containing compound selected from the group consisting of Bi₂O₃, bismuth carbonate, bismuth hydrogencarbonate, bismuth halide, Bi(C₆-C₁₄-aryl)₃, Bi(C₁-C₁₂-alkyl)₃ and metallic bismuth.

12. The method as claimed in claim 11, wherein the bismuth-containing compound is selected from the group consisting of Bi₂O₃, BiCl₃, Bi(C₆H₅)₃ and metallic bismuth.

13. The method as claimed in claim 11, wherein
i) the reaction is carried out under a protective atmosphere and/or in the presence of at least one solvent, and/or
ii) the reaction is conducted for at least 10 hours and/or at a temperature of at least 100° C., and/or
iii) following the reaction, volatile constituents are removed, the bismuth-containing catalyser is dried under reduced pressure and/or a recrystallization is carried out.

14. The method as claimed in claim 11, wherein a compound of the general formula (IIa) is prepared by reacting a corresponding compound (IIb), but in which R⁵ and/or R⁶ is H, with a lithium-containing compound, and the intermediate is subsequently reacted with a haloalkane to introduce the radicals R⁵ and/or R⁶ to obtain a compound according to the general formula (IIa).

15. A method of using the bismuth-containing catalyser as claimed in claim 1, the method comprising using the bismuth-containing catalyser for preparing compounds comprising a urethane group.

16. The bismuth-containing catalyser as claimed in claim 1 wherein x is equal to 3 and y and z are each equal to 0.

17. The bismuth-containing catalyser as claimed in claim 1, wherein R⁶ is C₆-C₁₀-alkyl.

18. The bismuth-containing catalyser as claimed in claim 7, wherein R⁶ is C₆-C₁₀-alkyl.

19. The method as claimed in claim 11, wherein the reaction is carried out under a protective atmosphere and/or in the presence of toluene or tetrahydrofuran.

20. The method as claimed in claim 11, wherein a compound of the general formula (IIa) is prepared by reacting a corresponding compound (IIb), but in which R⁵ and/or R⁶ is H, with n-butyllithium.
